(19) United States
(12) Patent Application Publication
(10) Pub. No.: US 2026/0224572 A1
(43) Pub. Date: Aug. 6, 2026

(54) TREATING CANCER WITH AN ADENOSINE A2A INHIBITOR

(71) Applicant: IMPETIS BIOSCIENCES LIMITED, Mumbai (IN)

(72) Inventors: Kasim A. Mookhtiar, Mumbai (IN); Siddhartha De, Mumbai (IN); Ashwinkumar V. Meru, Mumbai (IN)

(21) Appl. No.: 19/151,245

(22) PCT Filed: Jan. 25, 2024
(86) PCT No.: PCT/IB2024/050682

§ 371 Date: Jul. 25, 2025

Related U.S. Application Data
(60) Provisional application No. 63/441,090, filed on Jan. 25, 2023.

(51) Int. Cl. A61K 31/519; A61K 9/00; A61K 31/522; A61K 39/395

(52) U.S. Cl. CPC A61K 31/519; A61K 9/0053; A61K 31/522; A61K 39/3955

(57) ABSTRACT

Methods for treating cancers, including solid tumors, by administering the compound PNQ-370 (5-amino-3-[2-[4-[2-fluoro-4-(2-methoxyethoxy)phenyl]piperazin-1-yl]ethyl]-8-(2-furyl)-1-methyl-[1,2,4]triazolo[5,1-f]purin-2-one) to selectively antagonize the A2A receptor are described. The methods comprise orally administering PNQ-370 to a subject in need at particular doses. In an embodiment, the method comprises administering a second compound that has activity to inhibit/antagonize (i) an adenosine A2B receptor and/or (ii) a checkpoint programmed death 1 (PD-1) receptor.

### CROSS-REFERENCE TO RELATED APPLICATIONS

This application claims the benefit of U.S. Provisional Application No. 63/441,090, filed Jan. 25, 2023, incorporated herein by reference in its entirety.

### REFERENCE TO SEQUENCE LISTING, TABLE OR COMPUTER PROGRAM

The instant application contains a Sequence Listing which has been filed electronically in XML format and is hereby incorporated by reference in its entirety. The XML copy, created on Jan. 24, 2024, is named 108428-0097-8007WO00_SEQ.XML and is 2,657 bytes in size.

### TECHNICAL FIELD

The subject matter described herein relates to methods for treating cancers by administering the compound PNQ-370 (5-amino-3-[2-[4-[2-fluoro-4-(2-methoxyethoxy)phenyl]piperazin-1-yl]ethyl]-8-(2-furyl)-1-methyl-[1,2,4]triazolo[5,1-f]purin-2-one) to selectively antagonize the A_{2A} receptor.

### BACKGROUND

Adenosine is an intermediate for the synthesis of adenosine triphosphate (ATP), adenine, and adenylate. Extracellular adenosine acts as a signal molecule to activate adenosine receptors, which are G-protein coupled receptors. Four adenosine receptors have been found thus far: A₁ receptor, A_{2A} receptor, and A₃ receptor with high affinity for adenosine, and A_{2B} receptor with low affinity for adenosine. The A_{2A} receptor is widely distributed on the surface of most immune cells. The role of the adenosine-A_{2A} receptor pathway in the human body, in cancer and in other diseases such as Parkinson's disease, has made the development of effective and selective A_{2A} adenosine receptor antagonists desirable. Despite the desire and appeal for compounds that antagonize the A_{2A} receptor, such compounds to date have issues that prevent them from being used clinically, such as affinity for the A₁ adenosine receptor subtype or an undesirable pharmacokinetic parameter. Identification of compounds that specifically and strongly antagonize the A_{2A} receptor are desired.

### BRIEF SUMMARY

The following aspects and embodiments thereof described and illustrated below are meant to be exemplary and illustrative, not limiting in scope.

In one aspect, methods for treating cancer are provided. Methods for treating a solid tumor are also provided. The methods comprise orally administering a compound having the structure of Compound 1 below, to a subject in need at a dose of between about 1-30 or about 5-20 mg per day:

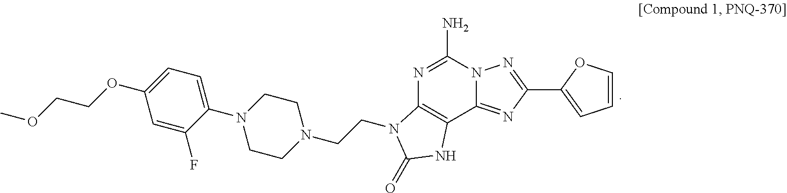 [SMILES: COCCOc1ccc(N2CCN(CCn3c(=O)[nH]c4c3nc(N)n3nc(-c5ccco5)nc43)CC2)c(F)c1]

In an embodiment, the method further comprises determining an A_{2A} receptor expression level of the solid tumor prior to orally administering PNQ-370.

In an embodiment, orally administering comprises orally administering as a monotherapy.

In an embodiment, the method further comprises surgical resection of the solid tumor, before, after or concurrent with orally administering PNQ-370.

In an embodiment, the method further comprises providing or receiving radiation treatment before, after or concurrent with orally administering PNQ-370.

In an embodiment, orally administering comprises orally administering the dose as a total daily dose divided into two portions.

In an embodiment, the solid tumor is associated with breast cancer or colon cancer.

In an embodiment, PNQ-370 is orally administered at dose of between about 0.5-15 mg given twice daily, whereby the orally administering provides a concentration of PNQ-370 in a solid tumor about 10 fold higher than a concentration in the plasma.

In an embodiment, the concentration of PNQ-370 in a solid tumor is about 10 fold higher than a concentration in the plasma within about 15 minutes, 30 minutes, 1 hour, 2 hours, or 3 hours after oral administration.

In an embodiment, the concentration of PNQ-370 in a solid tumor is about 10 fold higher than a concentration in the plasma for between about 1-4 hours after oral administration.

In an embodiment, the orally administering is with food.

In an embodiment, the solid tumor is castrate-resistant prostate cancer, non-small cell lung cancer, head and neck cancer, or renal cell cancer.

In an embodiment, the non-small cell lung cancer is squamous cell cancer.

In another aspect, a method for treatment is provided. The method comprises orally administering PNQ-370, and administering a second compound that (i) has activity to inhibit/antagonize an adenosine A_{2B} receptor, (ii) is an immune-oncologic agent, such as a checkpoint programmed death 1 (PD-1) receptor ligand monoclonal antibody (PD-L1 mAb) or CTLA4Ig, or (iii) is a cytotoxic chemotherapeutic agent.

In an embodiment, administering the second compound comprises administering concurrently with or subsequently to administering Compound 1.

In an embodiment, administering the second compound comprises administering enterally or parenterally.

In an embodiment, the second compound is an antagonist of the A_{2B} receptor.

In an embodiment, the second compound is Compound 2 or a prodrug thereof:

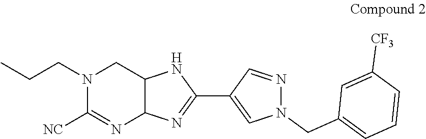 [SMILES: CCCN1CC2NC(c3cnn(Cc4cccc(C(F)(F)F)c4)c3)=NC2N=C1C#N]

### 6-Oxo-1-propyl-8-[1-(3-trifluoromethyl-benzyl)-1H-pyrazol-4-yl]-6, 7-dihydro-1H-purine-2-carbonitrile

In an embodiment, the second compound is an inhibitor of the PD-1 receptor.

In an embodiment, the second compound is an anti-PD-1 receptor antibody.

In an embodiment, the antibody is selected from ipilimumab, nivolumab and pembrolizumab.

In an embodiment, orally administering of Compound 1 is at a dose of between about 0.5-15 mg given twice daily.

In an embodiment, a dose of one or both of the compounds is determined based on an evaluation of receptor expression (density) of cancerous tissue in the patient. In an embodiment, treatment of the subject includes an evaluation of receptor expression (density) from a biopsy of the subject's cancerous tissue.

In an embodiment, the cancer is non-small cell lung cancer, triple negative breast cancer, renal cancer, melanoma, prostate cancer, endometrial cancer, salivary gland cancer, sinus cancer, parotid cancer, cervical cancer, or any other cancer that is found to express the A2_{A} receptor.

In an embodiment, the cancer is colorectal cancer, ovarian cancer, non-small cell lung cancer, triple negative breast cancer, prostate cancer, endometrial cancer, or pancreatic cancer.

In an embodiment, the methods further comprise determining an A_{2A} receptor expression level of cells associated with the cancer prior to orally administering Compound 1.

In an embodiment, the methods further comprise determining an A_{2B} receptor expression level of cells associated with the cancer or a checkpoint programmed death 1 (PD-1) receptor expression level of cells associated with the cancer prior to orally administering Compound 1, prior to administering the second compound, or both.

In addition to the exemplary aspects and embodiments described above, further aspects and embodiments will become apparent by reference to the drawings and by study of the following descriptions.

Additional embodiments of the present methods will be apparent from the following description, drawings, examples, and claims. As can be appreciated from the foregoing and following description, each and every feature described herein, and each and every combination of two or more of such features, is included within the scope of the present disclosure provided that the features included in such a combination are not mutually inconsistent. In addition, any feature or combination of features may be specifically excluded from any embodiment of the present disclosure. Additional aspects and advantages of the present disclosure are set forth in the following description and claims, particularly when considered in conjunction with the accompanying examples and drawings.

### BRIEF DESCRIPTION OF THE DRAWINGS

FIG. 1 is a graph of the mean plasma concentration, in μM, as a function of time, in hours, of compound PNQ-370 after administration to rats intravenously (3 mg/kg, closed circles), orally (10 mg/kg, open squares) and orally (5 mg/kg solution, close squares).

FIG. 2A is a bar graph showing A_{2A} receptor occupancy by compound PNQ-370 in rat brain striatum 3 hours after oral dosing to rats at doses of 0.1 mg/kg, 0.3 mg/kg and 1 mg/kg or 3 hours after oral dosing with preladenant at the same 3 doses.

FIG. 2B is a bar graph showing the time course of A_{2A} receptor occupancy by compound PNQ-370 and preladenant in rat brain striatum after oral dose of 1 mg/kg.

FIGS. 3A-3C are graphs showing the binding activity of PNQ-370 on human A_{2A} adenosine receptor (AdoR) in competitive radioligand binding assay. Preladenant and SYN-115 (tozadenant) were used as comparator compounds. Each graph shows the percent of binding to human A_{2A} AdoR in membranes over-expressing the receptor as a function of concentration of test compound, in log M, for PNQ-370 and SCH-442416 (a known antagonist to human A_{2A} AdoR) (FIG. 3A, closed circles and open circles, respectively), and for the comparator compounds preladenant and SYN-115 (FIGS. 3B and 3C). Data are presented as mean±SEM (of triplicates).

FIG. 4A is a schematic representation of the protocol described in Example 4 to determine binding off-rates of test compounds on a human A_{2A} adenosine receptor.

FIGS. 4B-4C are graphs showing the off-rate of PNQ-370 on human A_{2A} AdoR as a function of displacement by [3H]-ZM-241385 from two separate studies using a radioligand binding assay. Human A_{2A} AdoR over-expressing membranes were pre-incubated with PNQ-370 for 10 minutes followed by incubation in reaction buffer for varying time (with a wash at 30 minutes and every 1 hour). Membranes were then incubated with [3H]-ZM-241385, washed and the amount of radioligand retained after 15 minutes of incubation was determined. PNQ-370, preladenant, SYN-115 and MRS-1191 were used at 6 nM, 60 nM, 200 nM and 300 nM respectively.

FIGS. 4D-4E are graphs showing the off-rate of PNQ-370 on human A_{2A} AdoR as a function of displacement by [3H]-ZM-241385 in the absence (FIG. 4D) and presence of 3 μM 5′-N-ethylcarboxamidoadenosine (NECA) (FIG. 4E), using an assay and drug concentrations as described in FIGS. 4B-4C.

FIGS. 5A-5F are graphs from a Schild analysis carried out using homogeneous time-resolved fluorescence (HTRF)-based competitive immunoassay that detects the changes in secondary messenger cyclic adenosine monophosphate (cAMP) levels. HEK-293 cells overexpressing human A_{2A} AdoR were treated with increasing concentrations of 4-((2-carboxymethyl) phenyl) ethylamino)-5′-N-ethylcarboxamidoadenosine (CGS-21680) in presence or absence of different concentrations of PNQ-370 (FIG. 5A) preladenant (FIG. 5C) and SYN-115 (FIG. 5E) and the shift in EC₅₀ for the agonist was used to calculate dose ratio (DR). Log (DR-1) was plotted against antagonist concentrations to obtain Schild plots and calculate K_{B} for PNQ-370 (FIG. 5B), preladenant (FIG. 5D) and SYN-115 (FIG. 5F). The data presented is mean±SEM from at least 3 independent experiments each carried out in triplicates.

FIGS. 6A-6B are graphs showing the dissociation contestant (K_{D}) and density (concentration) of receptors in a sample of tissue (Bmax) determination A_{2A} AdoR antagonist ZM-241385 on human A_{2A} AdoR in a Scatchard analysis study.

FIGS. 6C-6E are graphs showing the effect of PNQ-370 on the Scatchard plot for ZM-241385 in a radioligand binding assay, where the ratio of bound to free A_{2A} AdoR antagonist ZM-241385 as a function of bound ZM-241385 were determined in absence or presence of different concentrations of PNQ-370 (FIG. 6C), preladenant (FIG. 6D) or SYN-115 (FIG. 6E).

FIGS. 7A-7C are graphs showing the effect of PNQ-370 on agonist-induced activation of human A_{2A} AdoR. Cells over-expressing A_{2A} AdoR were treated with increasing concentrations of PNQ-370 in presence of 10 nM A_{2A} AdoR CGS-21680 (K_{D}=2 nM). Preladenant and SYN-115 were used as comparator compounds. Change in cAMP level was measured using an HTRF based competitive immunoassay. Data are presented as mean±SEM (of triplicates).

### DETAILED DESCRIPTION

### I. Definitions

Various aspects now will be described more fully hereinafter. Such aspects may, however, be embodied in many different forms and should not be construed as limited to the embodiments set forth herein; rather, these embodiments are provided so that this disclosure will be thorough and complete, and will fully convey its scope to those skilled in the art.

Where a range of values is provided, it is intended that each intervening value between the upper and lower limit of that range and any other stated or intervening value in that stated range is encompassed within the disclosure. For example, if a range of 1 μm to 8 μm is stated, it is intended that 2 μm, 3 μm, 4 μm, 5 μm, 6 μm, and 7 μm are also explicitly disclosed, as well as the range of values greater than or equal to 1 μm and the range of values less than or equal to 8 μm.

The singular forms “a,” “an,” and “the” include plural referents unless the context clearly dictates otherwise. Thus, for example, reference to a “polymer” includes a single polymer as well as two or more of the same or different polymers, reference to an “excipient” includes a single excipient as well as two or more of the same or different excipients, and the like.

The term “about”, particularly in reference to a given quantity, is meant to encompass deviations of plus or minus five percent.

The compositions of the present disclosure can comprise, consist essentially of, or consist of, the components disclosed.

All percentages, parts and ratios are based upon the total weight of the topical compositions and all measurements made are at about 25° C. (room temperature), unless otherwise specified.

The phrase “pharmaceutically acceptable” is employed herein to refer to those compounds, salts, compositions, dosage forms, etc., which are within the scope of sound medical judgment suitable for use in contact with the tissues of human beings and/or other mammals without excessive toxicity, irritation, allergic response, or other problem or complication, commensurate with a reasonable benefit/risk ratio. In some aspects, “pharmaceutically acceptable” means approved by a regulatory agency of the federal or a state government, or listed in the U.S. Pharmacopeia or other generally recognized pharmacopeia for use in mammals (e.g., animals), and more particularly, in humans.

The term “pharmaceutically acceptable salt” embraces salts with a pharmaceutically acceptable acid or base. Pharmaceutically acceptable acids include both inorganic acids, for example hydrochloric, sulphuric, phosphoric, diphosphoric, hydrobromic, hydroiodic and nitric acid and organic acids, for example citric, fumaric, maleic, malic, mandelic, ascorbic, oxalic, succinic, tartaric, benzoic, acetic, methanesulphonic, ethanesulphonic, benzenesulphonic or p-toluenesulphonic acid. Pharmaceutically acceptable bases include alkali metal (e.g. sodium or potassium) and alkali earth metal (e.g. calcium or magnesium) hydroxides and organic bases, for example alkyl amines, arylalkyl amines and heterocyclic amines. Other include quaternary ammonium compounds wherein an equivalent of an anion (X−) is associated with the positive charge of the N atom. X− may be an anion of various mineral acids such as, for example, chloride, bromide, iodide, sulphate, nitrate, phosphate, or an anion of an organic acid such as, for example, acetate, maleate, fumarate, citrate, oxalate, succinate, tartrate, malate, mandelate, trifluoroacetate, methanesulphonate and p-toluenesulphonate. X− is preferably an anion selected from chloride, bromide, iodide, sulphate, nitrate, acetate, maleate, oxalate, succinate or trifluoroacetate. IN an embodiment, X− is chloride, bromide, trifluoroacetate or methanesulphonate.

The term “treating” is used herein, for instance, in reference to methods of treating cancer, and generally includes the administration of a compound or composition which reduces the frequency of, or delays the onset of, symptoms of a medical condition (e.g., cancer) in a subject relative to a subject not receiving the compound or composition. This can include reversing, reducing, or arresting the symptoms, clinical signs, and underlying pathology of a condition in a manner to improve or stabilize a subject's condition (e.g., regression of tumor growth).

The terms “inhibiting” or “reducing” are used in reference to methods to inhibit or to reduce tumor growth (e.g., decrease the size of a tumor) in a population as compared to a untreated control population.

By reserving the right to proviso out or exclude any individual members of any such group, including any sub-ranges or combinations of sub-ranges within the group, that can be claimed according to a range or in any similar manner, less than the full measure of this disclosure can be claimed for any reason. Further, by reserving the right to proviso out or exclude any individual substituents, analogs, compounds, ligands, structures, or groups thereof, or any members of a claimed group, less than the full measure of this disclosure can be claimed for any reason.

Throughout this disclosure, various patents, patent applications and publications are referenced. The disclosures of these patents, patent applications and publications in their entireties are incorporated into this disclosure by reference in order to more fully describe the state of the art as known to those skilled therein as of the date of this disclosure. This disclosure will govern in the instance that there is any inconsistency between the patents, patent applications and publications cited and this disclosure.

For convenience, certain terms employed in the specification, examples and claims are collected here. Unless defined otherwise, all technical and scientific terms used in this disclosure have the same meanings as commonly understood by one of ordinary skill in the art to which this disclosure belongs.

### II. Methods of Treatment

Methods for treating cancer, and in an embodiment, a solid tumor, are provided. The methods comprise administering a compound having the structure of Compound 1:

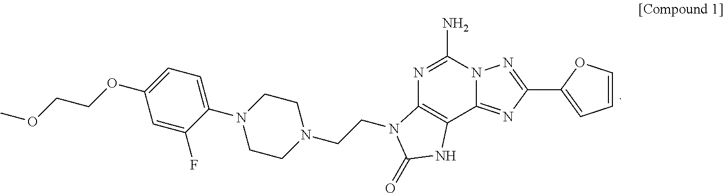 [SMILES: COCCOc1ccc(N2CCN(CCn3c(=O)[nH]c4c3nc(N)n3nc(-c5ccco5)nc43)CC2)c(F)c1]

Compound 1 is also referred to herein as PNQ-370. Its chemical name is 5-amino-3-[2-[4-[2-fluoro-4-(2-methoxyethoxy)phenyl]piperazin-1-yl]ethyl]-8-(2-furyl)-1-methyl-[1,2,4]triazolo[5,1-f]purin-2-one. Reference to “Compound 1” or PNQ-370 includes pharmaceutically acceptable salts, analogs, tautomeric forms, stereoisomers, geometrical isomers, polymorphs, hydrates, solvates, and metabolites thereof.

In embodiments, PNQ-370 is in the form of a pharmaceutical composition, for administration in accord with the methods described herein. The composition comprises PNQ-370 and a pharmaceutically-acceptable excipient. As mentioned above, PNQ-370 can be in base form, or can be a pharmaceutically acceptable salt, an analog, a tautomeric form, a stereoisomer, a geometrical isomer, a polymorph, a hydrate, a solvate, a metabolite, or a prodrug of Compound 1.

PNQ-370 is an antagonist of the A_{2A} receptor, which are expressed on immune cell types, including T lymphocytes, dendritic cells, natural killer cells. A_{2A} receptor activation on T cells and NK cells causes immunosuppression by reducing their proliferation, cytokine production and tumor killing activity. PNQ-370 as an antagonist of the A_{2A} receptor induces anti-tumoral responses in multiple types of cancer. As will be discussed herein in connection with Examples 1-7, PNQ-370 is potent and highly selective for the adenosine A_{2A} receptor. Its long receptor occupancy, particularly in the presence of high adenosine concentrations, allows for unique dosing and dosing regimens for cancer therapy. Additionally, it's binding affinity and selectivity for the A_{2A} receptor allow for unique dosing and dosing regimens for cancer therapy.

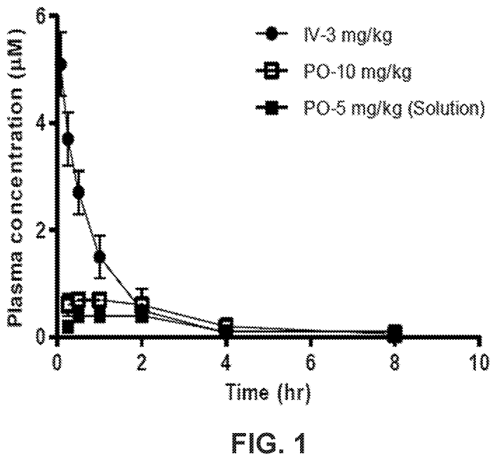

Example 1 describes study to investigate the pharmacokinetics profile of PNQ-370 following administration as an intravenous (3 mg/kg) and via oral gavage (10 mg/kg suspension or 5 mg/kg solution) to rats. Blood samples were collected at selected time points post-dosing for quantification of PNQ-370. Results are shown in Tables 1-1 and 1-2 and in FIG. 1. The data shows that following intravenous administration of PNQ-370 the mean systemic plasma exposure (AUC0-inf) and T_{1/2} were 4.8 μM/hr and 0.7 hr, respectively (Table 1-2). The concentration of PNQ-370 in plasma declined with time and it was eliminated completely after 4 hours post dose (FIG. 1). The mean apparent steady state volume of distribution (Vss) of PNQ-370 was 0.9 L/kg (Table 1-2), which is approximately equal to total body water normalized to body weight (0.7 L/kg) in rats. PNQ-370 showed a moderate clearance (CL) of approximately 20 mL/min/kg which was about 30% of normal hepatic blood flow in rats (Table 1-2).

With continuing reference to FIG. 1 and Table 1-2, after oral administration as a suspension formulation at 10 mg/kg dose, PNQ-370 showed mean Cmax and mean Tmax of 0. μM and 0.9 hour, respectively. The mean systemic exposure (AUC0-inf) was found to be 2.6 μM·hr. The plasma concentrations of PNQ-370 declined gradually with time and it was eliminated completely after 8 hours following dose administration (FIG. 1). The oral bioavailability of PNQ-370 in rats following suspension administration was estimated to be approximately 15% (Table 1-2).

After oral administration as a solution formulation at 5 mg/kg dose, PNQ-370 showed mean Cmax and mean Tmax of 0. μM and 1.0 hour, respectively. The mean systemic exposure (AUC0-inf) was found to be 1.8 μM/hour. The oral bioavailability of PNQ-370 in rats following solution administration was estimated to be approximately 19% (Table 1-2).

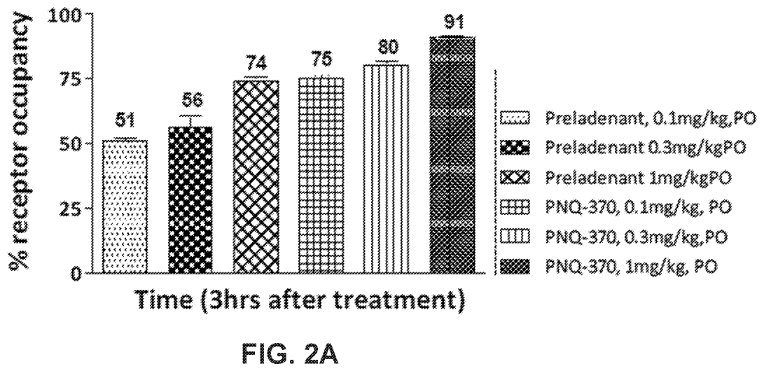

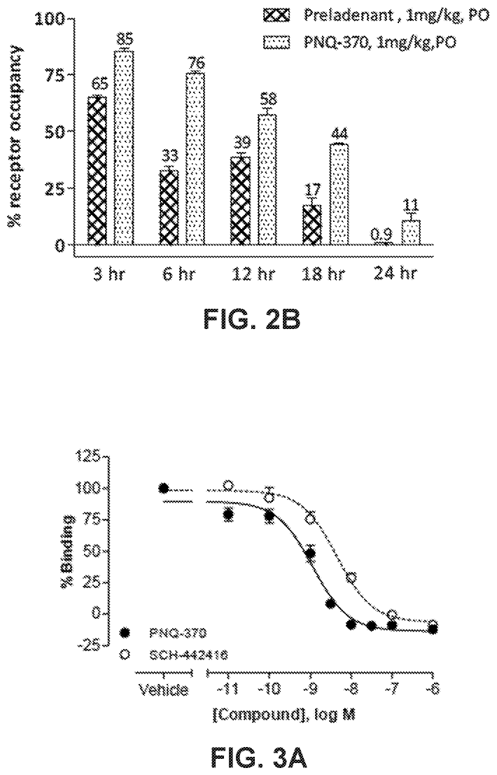

Example 2 describes a study of A_{2A} receptor occupancy of PNQ-370 in rat brain striatum after oral dosing by ex vivo receptor occupancy assay. Wistar rats were fasted overnight and were administered PNQ-370 or preladenant (at 0.1 mg/kg, 0.3 mg/kg and 1 mg/kg orally for dose response study and 1 mg/kg orally for time response study) or vehicle. At specified time points after dosing, animals were euthanized, brains were removed and striata tissue dissected, processed and analyzed as described in Example 2. Results are shown in FIGS. 2A-2B, and Tables 2-2 and 2-3 of Example 2. PNQ-370 showed dose dependent receptor occupancy of 75% at 0.1 mg/kg, 80% at 0.3 mg/kg and 91% at 1 mg/kg at 3 hours post dosing. The reference compound preladenant showed receptor occupancy of 51% at 0.1 mg/kg, 56% at 0.3 mg/kg and 74% at 1 mg/kg dose. In the time course study, PNQ-370 at 1 mg/kg showed receptor occupancy of 85% at 3 hours, 76% at 6 hours, 58% at 12 hours, 44% at 18 hours and 11% at 24 hours. Reference compound preladenant after oral dosing of rats at 1 mg/kg showed receptor occupancy of 65% at 3 hours, 33% at 6 hours, 39% at 12 hours, 17% at 18 hours and 0.9% at 24 hours.

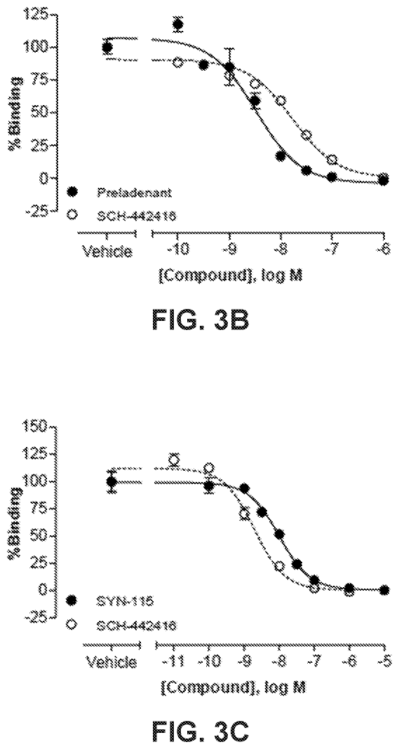

In Example 3, a study to determine the binding affinity of PNQ-370 to human A_{2A} adenosine receptor (AdoR) using a competitive radioligand binding assay is described. Membrane fractions were prepared from HEK-293 cells over-expressing human A_{2A} AdoR and used in radioligand binding assays using [3H]-ZM-241385 (A_{2A} AdoR antagonist) as radioligand. The results for this study showed that PNQ-370 binds to human A_{2A} AdoR at IC50 of 1.2±0.45 nM (binding affinity (Ki)=0.60±0.24 nM). Results are shown in FIGS. 3A-3C and Table 3-1. Preladenant and SYN-115 (tozadenant, CAS No. 870070-55-6) were used as comparator compounds. FIGS. 3A-3C show that increasing concentrations of test compounds displaces the specific binding of radiolabeled selective human A_{2A} AdoR antagonist [3H]-ZM-241385 (KD=1.1 nM). PNQ-370 (closed circles, FIG. 3A) binds to human A_{2A} AdoR at IC₅₀ of 1.2±0.45 nM (Ki=0.60±0.24 nM). Assay standard SCH-442416, known antagonist to human A_{2A} AdoR, binds at IC₅₀ of 6.1±2.6 nM (Ki=3.1±1.4 nM) (open circles, FIG. 3A). Preladenant binds to human A_{2A} AdoR at IC₅₀=2.7±0.4 nM (Ki=1.3±0.2 nM) (n=6) (FIG. 3B, closed circles). SYN-115 binds to human A_{2A} AdoR at IC₅₀=18.7±6.4 nM (Ki=9.9±3.5 nM) (FIG. 3C, closed circles).

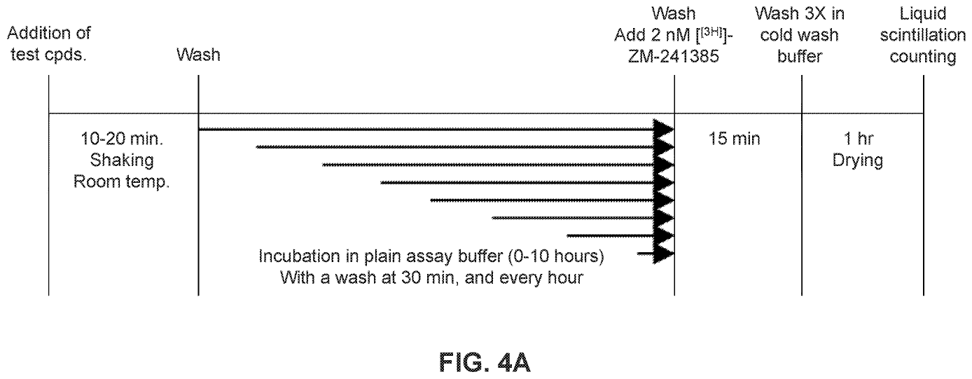

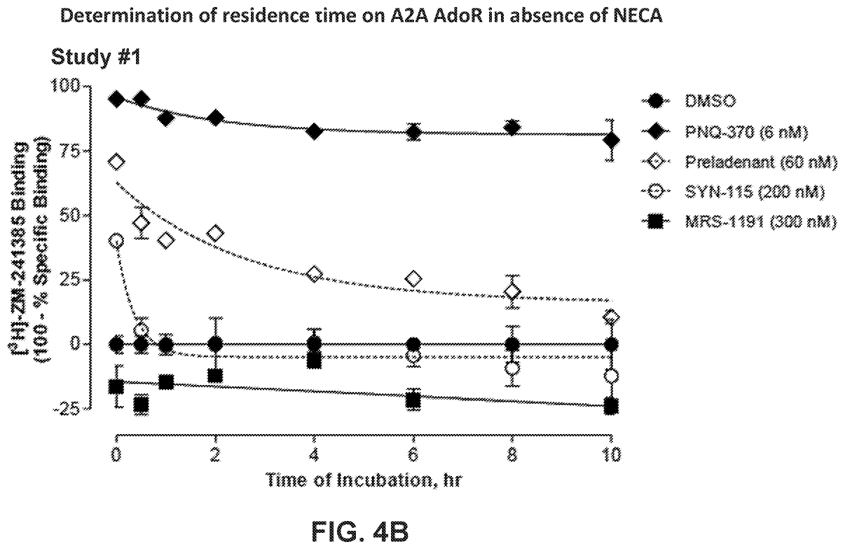

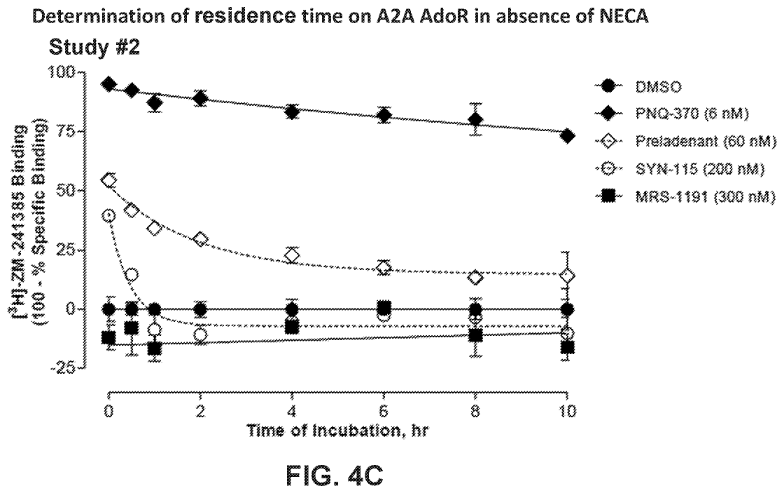

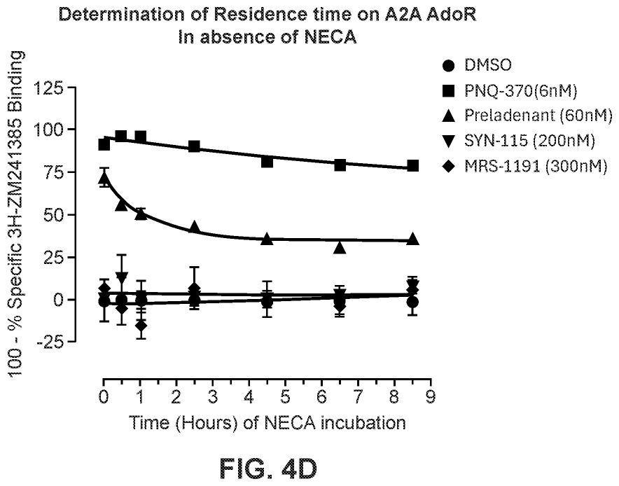

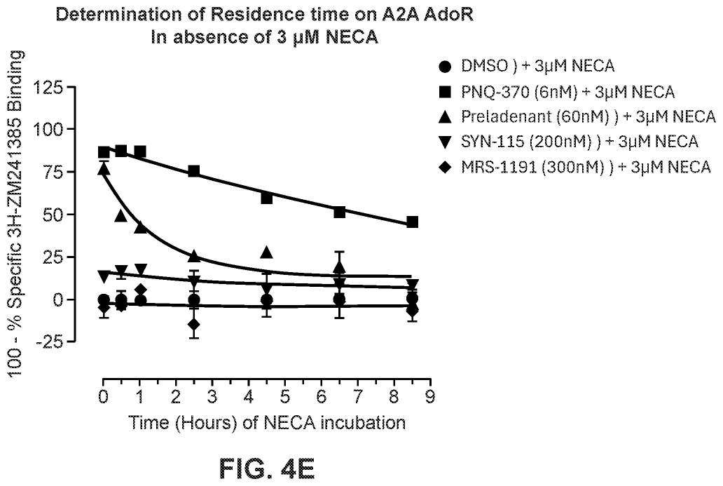

Since the antagonist PNQ-370 showed longer brain occupancy than the comparator preladenant in rat brain receptor occupancy assays on A_{2A} AdoR (Example 2), another study was done to determine whether this longer brain occupancy was a function of increased residence time of PNQ-370 at the A_{2A} AdoR. Example 4 describes this study, where binding assays were carried out with membranes over-expressing human A_{2A} AdoR to measure the K_{OFF} of both compounds. In addition to using PNQ-370 and preladenant, SYN-115 and MRS-1191 (an A₃ AdoR antagonist, CAS No.: 185222-90-6, >300-fold selective over A_{2A} AdoR) were also evaluated. With reference to FIGS. 4B-4C, PNQ-370 exhibited a long K_{OFF} on human A_{2A} AdoR and even after 10 hours of repeated washing, the compound did not appreciably dissociate from the receptor. In contrast, preladenant showed a steady rate of decline in binding, indicating that it has a quicker off-rate compared to PNQ-370. These observations are consistent with those seen in the brain receptor occupancy assay.

SYN-115 appears to have significantly different properties compared to PNQ-370 and preladenant, as observed with its much faster K_{OFF}. Almost 100% displacement of SYN-115 binding by ZM-241385 was observed within 1 hour. Despite using a saturating dose and longer pre-incubation with SYN-115, 100% inhibition of radioligand binding was not observed even at the earliest time point evaluated (FIGS. 4B-4C).

A similar study as set forth in Example 4 was performed, where the off-rates from human A_{2A} AdoR of the compounds as a function of displacement by [3H]-ZM-241385 was determined in the absence (FIG. 4D) and presence of 3 μM 5′-N-ethylcarboxamidoadenosine (NECA, FIG. 4E).

This study provided an assessment of the off-rate of PNQ-370, and the comparator compounds preladenant and SYN-115, and MRS-1191 (a negative control), in an adenosine-containing environment, as is the case in a tumor microenvironment. The data is shown in FIGS. 4D-4E. The K_{off} of greater than about 10 hours of PNQ-370 from human A_{2A} AdoR is maintained in the presence of NECA (FIG. 4D), evidencing that PNQ-370 has a K_{off} of at least about 10 hours in an adenosine-containing environment. As seen in FIG. 4E, the residence time of PNQ-370 on the human A_{2A} AdoR was longer than preladenant (triangles), SYN-115 (inverted triangles), and MRS-1191 (diamonds). Thus, PNQ-370 did not appreciably dissociate from the human A_{2A} AdoR over a 10 hour time period and in the presence of NECA (3 μM), whereas preladenant had a steady rate of decline in binding to the receptor with a 50% loss of binding within 1 hour in the presence of NECA (3 μM).

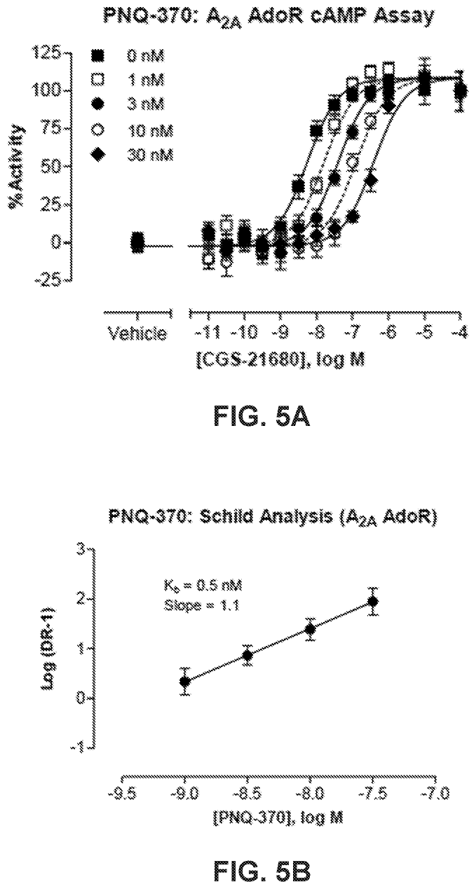

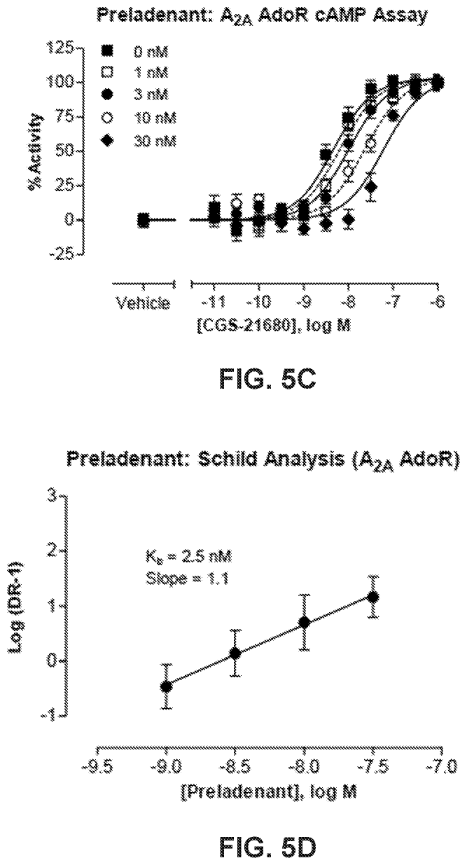

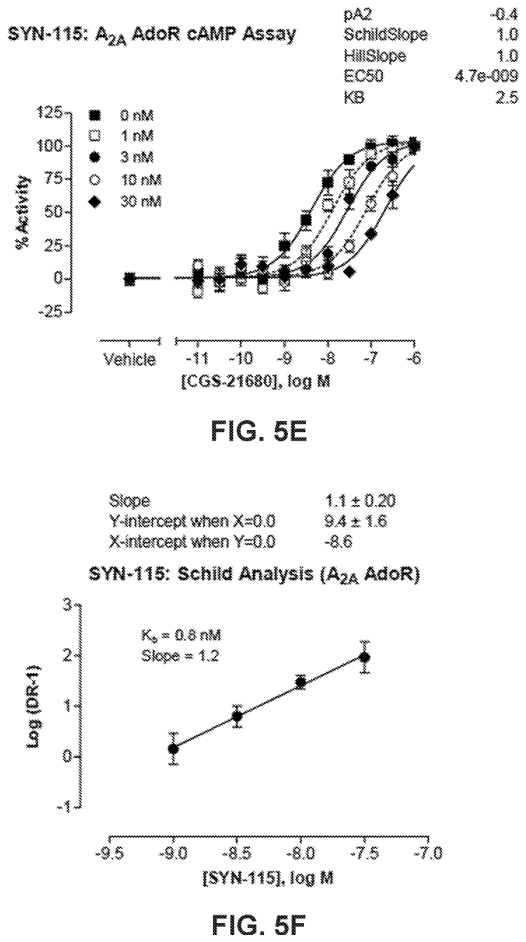

Based on the K-off studies carried out with PNQ-370, it was indicated that there is a significant difference between the residence time of PNQ-370 in comparison to comparator preladenant and SYN-115 for human A_{2A} AdoR. To further understand the mechanism of antagonism, the antagonists were evaluated for Schild regression as a function of Gs signaling, i.e. change in secondary messenger cAMP levels as described by Hodgson et. al. (J. Pharmacology and Experimental Therapeutics, 330(1): 294-303 (2009)). Example 5 describes a study using Schild regression analysis, which is a pharmacological method of receptor classification, wherein the dose effect curve for an agonist is determined in the presence of various concentrations of a competitive antagonist. Results are shown in FIGS. 5A-5F. CGS-21680 dose-dependently induced activation of human A_{2A} AdoR, indicated as a function of increased cAMP levels. A clear rightward shift in the CGS-21680 concentration response curve was obtained in presence of varying concentrations of PNQ-370, preladenant and SYN-115. The affinity estimates for PNQ-370 were similar to preladenant and SYN-115. The KB value for PNQ-370 was determined to be 0.5±0.04 nM. The resultant Schild plot was linear, with slope not significantly different from unity, indicating competitive antagonism. These results suggested that PNQ-370 functions as antagonist and competitively inhibits human A_{2A} AdoR at the agonist binding site. The Schild plots for comparator compounds preladenant and SYN-115 were also linear with slope not significantly different from unity. KB values for preladenant and SYN-115 were, respectively, 2.5±0.99 nM and 0.8±0.3 nM.

These binding studies to measure off-rates indicated that there is a significant difference between the residence time of PNQ-370, preladenant and SYN-115 at human A_{2A} AdoR. The Schild regression analyses carried out with HTRF based cAMP functional assay suggested that there is competitive antagonism at the functional level, leading to inhibition of CGS-21680-mediated activation of human A_{2A} AdoR. To understand whether the differences in residence time were simply a function of Koff, or whether differences in the mechanism of binding may also be contributing factors, the test compounds were subjected to a Scatchard analysis in a competitive radioligand binding assay.

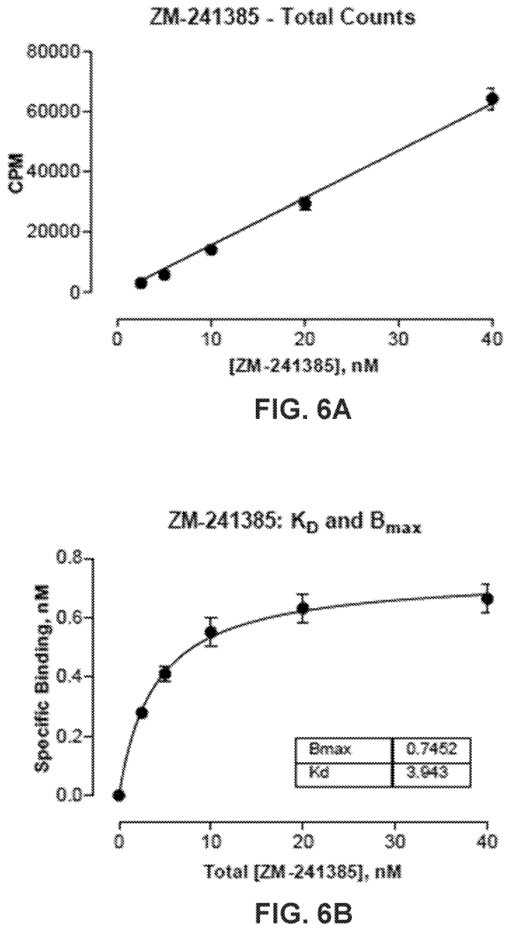

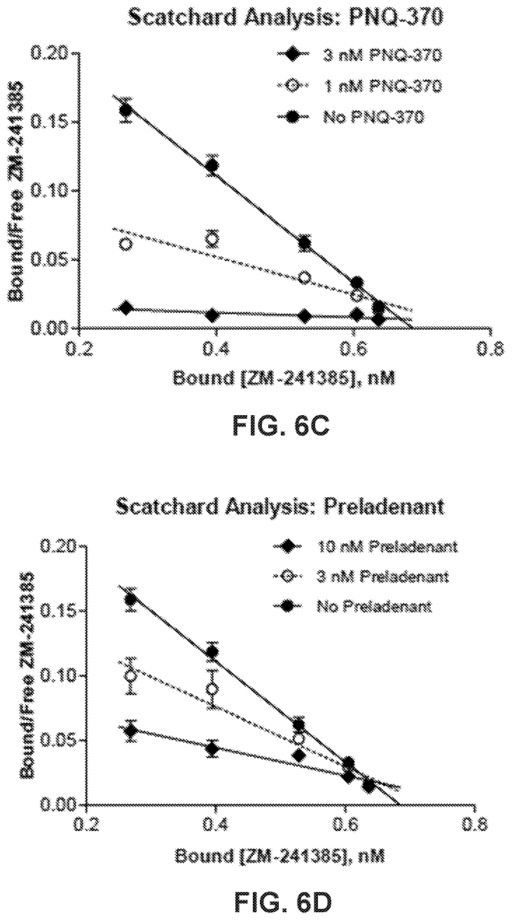

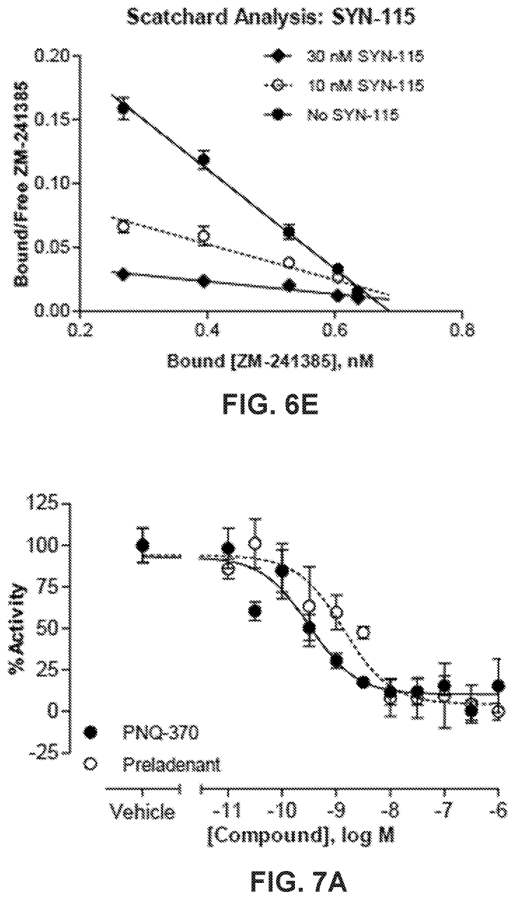

Example 6 describes the Scatchard analysis study. Binding mechanism of PNQ-370 was determined by evaluating its effect on the Scatchard plot of A_{2A} AdoR antagonist ZM-241385. Preladenant and SYN-115 were used as comparator compounds. Membranes expressing human A_{2A} AdoR (5 μg/well/100 μL) were incubated with increasing concentrations of A_{2A} AdoR antagonist ZM-241385, supplemented with [3H]-labeled ZM-241385 for detection in presence or absence of PNQ-370, preladenant or SYN-115 in reaction buffer. Specific binding in nM was determined from the ratio of total counts (no filtration or removal of any ligand) to the counts retained on the membrane (after correcting for nonspecific binding). Results are shown in FIGS. 6A-6E and Table 6-1.

FIGS. 6A-6B show the K_{D} and Bₘₐₓ for ZM-241385 were found to be 3.9 and 0.75 nM, respectively. In a Scatchard analysis, the slope of the line reflects the KD, and the intercept on the X-axis when Y=0 reflects the Bmax. As seen FIGS. 6C-6E competition with PNQ-370, preladenant and SYN-115 changes the KD of ZM-241385 at the human A_{2A} AdoR receptor, without appreciably changing the Bmax. These results indicate that the binding of PNQ-370 as well as preladenant and SYN-115 is competitive with respect to ZM-241385.

Example 7 describes a study that investigated the functional activity of PNQ-370 on (4-((2-carboxymethyl) phenyl) ethylamino)-5′-N-ethylcaboxamidoadenosine (CGS-21680)-induced activation of human A_{2A} AdoR in HEK-293 cells overexpressing A_{2A} AdoR in an in vitro multi-well plate assay. As described in Example 7, Human A_{2A} AdoR was cloned and stably expressed in HEK-293 cells. The cells were treated with adenosine deaminase, harvested and distributed at a density of 3750 cell/25 μL/well in cell media. The cells were incubated with incubated with increasing concentrations of the test compounds for 15 minutes, followed by treatment with the specific A_{2A} AdoR agonist CGS-21680 (final concentration 10 nM, Tocris) for 30 minutes. The levels of levels of cAMP were determined.

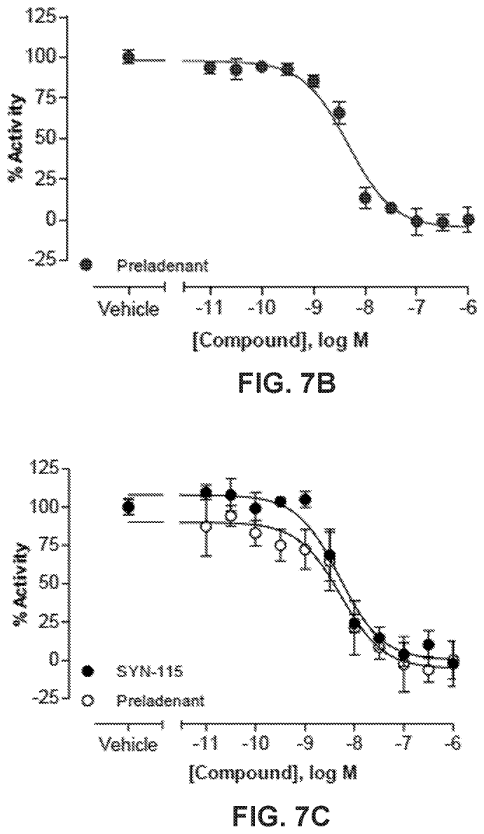

The results are shown in FIGS. 7A-7C, and Table 7-1 in Example 7. PNQ-370 inhibits CGS-21680-induced activation of human A_{2A} AdoR at IC₅₀ of 0.4±0.1 nM (Ki=0.062±0.02 nM), as seen in FIG. 7A. The assay standard preladenant, a known antagonist of human A_{2A} AdoR, inhibits CGS-21680-induced A_{2A} AdoR activity at IC₅₀ of 5.1±2.4 nM (Ki=0.7±0.3 nM). Preladenant and SYN-115 were evaluated as comparator compounds in separate experiments. Preladenant inhibits CGS-21680-induced A_{2A} AdoR activation at IC50=5.2±1.2 nM (Ki=0.7±0.2 nM), as seen in FIG. 7B. SYN-115 inhibits CGS-21680-induced A_{2A} AdoR activation at an IC50=7.0±3.1 nM (Ki=0.9±0.4 nM), as seen in FIG. 7B. The results for this study showed that PNQ-370 inhibits CGS-21680-induced activation of human A_{2A} AdoR at IC50 of 0.4±0.1 nM (Ki=0.062±0.02).

Accordingly, PNQ-370 is potent and highly selective for the adenosine A_{2A} receptor. Its long receptor occupancy, particularly in the presence of high adenosine concentrations, allows for unique dosing and dosing regimens for cancer therapy. Additionally, its binding affinity and selectivity for the A_{2A} receptor allow for unique dosing and dosing regimens for cancer therapy. These properties provide for a method for treating cancer, including a solid tumor associated with a cancer, by orally administering PNQ-370 at a daily dose of between about 0.1-37 mg, 0.1-35 mg, 0.5-35 mg, 0.5-30 mg, 1-35 mg, 1-30 mg, 2-35-mg, 2-30 mg, 2-25 mg, 2-20 mg, 2-10 mg, 5-35 mg, 5-30 mg, 5-25 mg, or 5-20 mg. In an embodiment, a method of treating cancer, including a cancer that is a solid tumor, comprises orally administering the daily dose of PNQ-370 once per day. In embodiments, the daily dose is administered in two equal portions (e.g., bis in die (bid)). In other embodiments, each portion or at least one of the portions is administered with food.

The studies in Examples 1-7 demonstrate that PNQ-370 is a potent antagonist for the A_{2A} receptor with superior selectivity for the receptor, as shown by the data in Tables 1A-1B.

TABLE 1A
Potency and Selectivity of PNQ-370
Potency (functional inhibition)

| Receptor | Ki (nm) | Selectivity |
|---|---|---|
| A2A | 0.065 | 1 |
| A1 | 10,000 | >150,000x |
| A2B | 100 | >1,400x |
| A3 | 10,000 | >150,000x |

TABLE 1B
Binding Affinity of PNQ-370 and other comparative compounds
Binding Affinity

| Drug | A2AR | A1R | A2BR | A3R |
|---|---|---|---|---|
| PNQ 370 | 0.68 | 700 | 2000 | 10,000 |
| CPI-444 | 3.52 | 54 | 531 | 693 |
| AZD4635 | 1.58 | 0.1(1) | N/A | N/A |
| Vipadenant(2) | 1.3 | 48 | 52 | 773 |
| Istradefylline | 36 | 79 | 50 | 83 |

PNQ-370 has an extended residence time on the receptor compared to other A2A receptor antagonists (See, FIG. 4B). And, the extended residence time of PNQ-370 relative to other A2A receptor antagonists is maintained in an adenosine-containing environment (FIG. 4C), as is the case for tumor microenvironments. The long residence time provides for a prolonged pharmacodynamic effect of greater than or at least about 8, about 9, or about 10 hours after administration. Its slow off rate from the receptor allows for an extended pharmacodynamic effect of at least about 8 hours, 9 hours or 10 hours The long pharmacodynamic effect permits PNQ-370 to be administered at a dose lower than other A2A receptor antagonists and/or at a frequency lower than other A2A receptor antagonists.

In an embodiment, the methods described herein further comprise determining or measuring receptor expression (density) from a biopsy of the subject's cancerous tissue. The determining can include determining density of A_{2A} receptors on the biopsy and/or determining relative concentrations of A_{2A}, A_{2B}, A₁ and/or A₃ receptors on the biopsy. The type and/or density of receptors may be used to determine dose of PNQ-370 and/or to determine whether to treat the subject with PNQ-370 as a monotherapy or in combination with one or more second therapeutic agents. In an embodiment, the method comprise a method for treating cancer, including a solid tumor, by administering PNQ-370 and administering a second therapeutic agent. In an embodiment, the second therapeutic agent is a compound that has activity to inhibit/antagonize (i) an adenosine A_{2B} receptor or (ii) a checkpoint programmed death 1 (PD-1) receptor and/or PD-1 protein. The second therapeutic agent can be administered concurrently with or subsequently to administering PNQ-370. The second therapeutic agent can be administered parenterally or enterally. In an embodiment, the second therapeutic agent is a compound that is an antagonist of the A_{2B} receptor, such as Compound 2. In an embodiment, the second compound is an inhibitor of the PD-1 receptor or an inhibitor of PD-1. Exemplary agents are ipilimumab, nivolumab and pembrolizumab.

The methods described find use, in embodiments, for treating non-small cell lung cancer, triple negative breast cancer, renal cancer, melanoma, prostate cancer, endometrial cancer, salivary gland cancer, sinus cancer, parotid cancer, or cervical cancer. In an embodiment, the cancer is colorectal cancer, ovarian cancer, non-small cell lung cancer, triple negative breast cancer, prostate cancer, endometrial cancer, or pancreatic cancer.

In an embodiment, a method for treating tumors characterized by hypoxia. Hypoxia has various forms—acute, chronic, toxic and systemic ones. In cancers, toxic form of hypoxia is not significant. Acute hypoxia is perfusion hypoxia, while chronic hypoxia is characterized as diffusion hypoxia, systemic hypoxia in cancer patients is also a frequent event. Collectively, cancer hypoxia is a combination of acute, chronic and systemic forms of hypoxia, which drives tumor progression and can cause resistance to various therapeutic modalities (Sebestydn, A. et al., Pathology & Oncology Research, Vol. 27, No. 1, 2021). Tumor hypoxia can be measured, for example, with polarographic electrodes or can be ascertained from the literature (Walsh, J. et al., Antioxidants & Redox Signaling, Vol. 21, No. 10, p. 1516 (2014)). Hypoxia increases adenosine levels as a result of cell death and also induces the expression of the adenosine A2A and A2B receptors. In an embodiment, a method of treating a solid tumor that is a hepatocellular carcinoma, a lung adenocarcinoma, colorectal cancer, or a renal cell carcinoma is provided. In other embodiments, the solid tumor associated with head and neck cancer, lung cancer, breast cancer, cervical cancer, renal cancer, cervical squamous cell cancer, prostate cancer, soft tissue sarcoma, or brain cancer. In an embodiment, the tumor is associated with triple negative and chemotherapy-resistant breast cancer. In an embodiment, the tumor is a glial tumor, rectal adenocarcinoma, or pancreatic adenocarcinoma. In embodiments, treatment of the solid tumor by oral administration of PNQ-370 is contemplated.

In other embodiments, a method for treating a solid tumor refractory to at least one prior therapy for treating the solid tumor. In an embodiment, the prior therapy is with a checkpoint inhibitor. In other embodiments, the refractory solid tumor is a solid tumor associated with head and neck cancer, lung cancer, breast cancer, cervical cancer, prostate cancer, soft tissue sarcoma, or brain cancer. In another embodiment, a method for treating a cancer, including a cancer that presents as a solid tumor, comprises determining a gene expression signature of adenosine signaling of the cancer or the tumor. Based on the adenosine expression signature, treatment with PNQ-370 alone or in combination with another therapy is recommended or provided.

### III. Examples

The following examples are illustrative in nature and are in no way intended to be limiting.

### Example 1

### Pharmacokinetic Study

The objective of this study was to investigate the pharmacokinetics profile of PNQ-370 following IV bolus and PO gavage administration in male Wistar rat (weighing 220±25 g). The study was conducted in three groups of animals each containing 4 animals.
Group 1: 3 mg/kg intravenous bolus (IV) solution dose of PNQ-370
Group 2: 10 mg/kg oral (PO) suspension dose of PNQ-370
Group 3: 5 mg/kg oral (PO) solution dose of PNQ-370

Animals in group of 4 were housed in numbered cages. The cage card identified each group with study number, animal group identification number, species, strain and gender. Animals in each group were identified by marking on the tail using a permanent marker. Animals were provided standard rodent chow and filtered water ad libitum. All animals were fasted for approximately 10-12 hours prior to dose administration and 2 hours post-dose.

Intravenous (IV) formulation: The solution formulation of PNQ-370 was prepared on the day of dosing (i.e. 0.6 mg/mL for IV and 0.5 mg/mL for PO solution). The composition of the vehicle was:
a. N-Methylpyrrolidone (NMP) 10% (v/v)
b. Cremophor EL (CrEL) 10% (v/v)
c. Polyethylene glycol-300 (PEG-300) 10% (v/v)
d. 0.1M ammonium acetate buffer (pH: 4.2) q.s.

An accurately weighed amount of PNQ-370 was transferred into a polypropylene tube and dissolved in the required volume of NMP. CrEL and PEG-300 were added subsequently with thorough mixing on a vortex mixer. The volume was made up with filtered 0.1 M ammonium acetate buffer with continuous vortex mixing.

Oral formulation: The formulation was prepared on the day of dosing as a suspension at 1 mg/mL concentration for 10 mg/kg dose. The required amount of PNQ-370 was weighed and transferred into a mortar and triturated after adding required amount of Tween 80® (final concentration 1% v/v). Then required volume of 0.5% sodium carboxymethyl cellulose (Na CMC) was further added in small aliquots to mortar and triturated with pestle to make a uniform suspension formulation of desired strength.

Animals in Group 1 received 3 mg/kg dose of PNQ-370 as an IV bolus (formulation strength: 0.6 mg/mL; dose volume: 5 mL/kg) via the tail vein. Animals in Group 2 received 10 mg/kg dose (suspension) via oral gavage (formulation strength: 1 mg/mL; dose volume: 10 mL/kg). Animals in Group 3 received 5 mg/kg dose (solution) via oral gavage (formulation strength: 0.5 mg/mL; dose volume: 10 mL/kg).

Blood samples (about 0.2 mL each via retro-orbital plexus) were collected at 0.083 (IV only), 0.25, 0.5, 1, 2, 4, 8 and 24 hour post-dose into labeled micro-centrifuge tubes containing K₂EDTA as an anticoagulant (25 μL of 20% (w/v) solution per mL of blood). The exact time of sampling with respect to dosing was recorded for individual animal. The collected blood samples were centrifuged at 4° C. at 6000 rpm for 5 minutes and obtained plasma was separated into labeled micro-centrifuge tubes and stored frozen below −70° C. until bioanalysis.

Quantitative bioanalysis of PNQ-370 in the plasma samples was performed using fit-for-purpose liquid chromatography tandem mass spectrometry (LC-MS/MS) method developed for the quantification of PNQ-370. Plasma samples were analyzed following protein precipitation of thawed plasma samples with acetonitrile containing internal standard (in house compound). Calibration range used for quantification of samples was from 10 ng/mL to 5000 ng/mL (r2=0.9974). The lower limit of quantitation was 10 ng/mL (0.02 μM).

Plasma pharmacokinetic parameters were calculated using the non-compartmental analysis tool of WinNonlin Professional software (Version 5.2.1). Pharmacokinetic parameters were determined from individual animals in each group. The area under the concentration versus time curve (AUC0-t and AUC0-inf), elimination half-life (T½), plasma clearance (CL) and steady state volume of distribution (Vss) were calculated from IV group. The peak plasma concentration (Cmax), time to achieve Cmax (Tmax), AUC0-t, AUC0-inf, and oral bioavailability (% F) were calculated from the PO groups.

TABLE 1-1
Plasma concentrations (μM) of PNQ-370 in Wistar
rats following single IV dose of 3 mg/kg and PO doses
of 10 and 5 mg/kg (mean ± SD)

| | | | |
|---|---|---|---|
| | IV | PO | PO |
| Time | 3 mg/kg | 10 mg/kg | 5 mg/kg |

| | |
|---|---|
| (hr) | Concentration in μM (mean ± SD) |

| | | | |
|---|---|---|---|
| 0.083 | 5.1 ± 0.6 | NA | NA |
| 0.25 | 3.7 ± 0.5 | 0.6 ± 0.2 | 0.2 ± 0.04 |
| 0.5 | 2.7 ± 0.4 | 0.7 ± 0.1 | 0.4 ± 0.04 |
| 1 | 1.5 ± 0.4 | 0.7 ± 0.1 | 0.4 ± 0.1 |
| 2 | 0.5 ± 0.1 | 0.6 ± 0.3 | 0.4 ± 0.07 |
| 4 | 0.08 ± 0.02 | 0.2 ± 0.1 | 0.12 ± 0.09 |
| 8 | BLQ | 0.06 ± 0.03 | 0.07 ± 0.03 |
| 24 | — | BLQ | BLQ |

BLQ: below limit of quantitation (0.02 μM or 10 ng/mL)

TABLE 1-2
Pharmacokinetic parameters (mean ± SD) of PNQ-370 in
male Wistar rats following intravenous and oral administration

| Route | | | | | | | | |
|---|---|---|---|---|---|---|---|---|
| (Dose | Tₘₐₓ | Cₘₐₓ | AUC₀₋ₜ | AUC_{0-inf} | T_{1/2} | CL | V_{SS} | |
| mg/kg) | (hr) | (μM) | (μM · hr) | (μM · hr) | (hr) | (mL/min/kg) | (L/kg) | F (%) |
| IV (3) | NA | NA | 4.7 ± 0.7 | 4.8 ± 0.7 | 0.7 ± 0.05 | 19 ± 3 | 0.9 ± 0.1 | — |
| PO (10) | 0.9 ± 0.8 | 0.8 ± 0.2 | 2.4 ± 1.0 | 2.6 ± 1.1 | | | | 15 ± 3 |
| PO (5) | 1.0 ± 0.7 | 0.4 ± 0.05 | 1.5 ± 0.4 | 1.8 ± 0.5 | | | | 19 ± 5 |

NA = not applicable

### Example 2

### Receptor Occupancy

The objective of this study was to determine A_{2A} receptor occupancy of PNQ-370 in rat brain striatum after oral dosing by ex vivo receptor occupancy assay.

PNQ-370 was prepared at specified doses in 0.5% Tween-80+0.5% CMC, q.s., 3 ml/kg, for oral (PO) administration.

Ex Vivo Receptor Occupancy Assay: Wistar rats were fasted overnight and were administered PNQ-370 or preladenant (at 0.1 mg/kg, 0.3 mg/kg and 1 mg/kg, PO for dose response study and 1 mg/kg, PO for time response study) or Vehicle (0.5% Tween-80+0.5% CMC, q.s., 3 ml/kg, PO). At specified time points after dosing, animals were euthanized with isofurane before decapitation. The brains were removed and striata dissected and collected. 50 mg of tissue was collected in 100 μl of assay buffer (20 mM Tris pH7.4, 120 mM NaCl, 5 mM KCl, 10 mM MgCl2 and 1 mM CaCl2)). Tissue was titurated nicely using pipetteman (~15 min/tube) and prepared smooth homogenized sample. To the homogenate adenosine diaminase (ADA) was added at a final concentration of 30 u/ml and incubated on shaker at room temperature for 60 minutes. Protein estimation of the homogenized striatal tissue sample was carried out using Bradford assay method (Sigma Cat #B6916).

Binding assay was set up in triplicates using equal concentration (70 g/reaction) of the homogenate protein and 2 nM of ZM-241385[3H]— (American Radiolabelled Chemicals, Cat No. ART-0884, specific activity 50 Ci/mmol). For non-specific count measurement 1 mM of standard antagonist SCH-442416 was added to binding reaction with vehicle treated striatal homogenate. The reaction mixtures were incubated for 60 minutes at room temperature (about 25° C.). The membrane receptor/ligand complex was collected onto a 96-well glass fiber filter mat (Wallac Cat #1450-421) under vacuum, washed free of unbound ligand with cold 20 mM Tris pH7.4 buffer, and allowed to dry at 55° C. To the dried filter mat 15 L/well of scintillation fluid (Optiphase supermix, Perkin Elmer Cat No: 1200-439) was added and fitted to filter mat 96 well holder and counted in MicroBeta Trilux plate counter.

Counts per minute from vehicle control group (specific binding) was considered as zero percent occupancy and nonspecific binding (1 mM of standard A_{2A} antagonist SCH-442416 in the binding reaction) was considered 100% receptor occupancy. Counts from reaction of compound treated tissue reactions was normalized to dilution factor and calculated percent A_{2A} receptor occupancy was plotted using Graphpad Prizm software.

The treatment groups are indicated in Table 2-1.

TABLE 2-1
Treatment Groups

| Time after | | Number of |
|---|---|---|
| dosing | Treatment | animals |
| 3 hours | PNQ-370, 1 mg/kg | 4 |
| | PNQ-370, 1 mg/kg | 4 |
| | Vehicle | 4 |
| 6 hours | PNQ-370, 1 mg/kg | 5 |
| | PNQ-370, 1 mg/kg | 5 |
| | Vehicle | 5 |
| 12 hours | PNQ-370, 1 mg/kg | 5 |
| | PNQ-370, 1 mg/kg | 5 |
| | Vehicle | 5 |
| 18 hours | PNQ-370, 1 mg/kg | 5 |
| | PNQ-370, 1 mg/kg | 5 |
| | Vehicle | 5 |
| 24 hours | PNQ-370, 1 mg/kg | 5 |
| | PNQ-370, 1 mg/kg | 5 |
| | Vehicle | 5 |

The test compound PNQ-370 showed dose dependent receptor occupancy of 75% at 0.1 mg/kg, 80% at 0.3 mg/kg and 91% at 1 mg/kg at 3 hours. The standard compound preladenant (SCH-442416) showed receptor occupancy of 51% at 0.1 mg/kg, 56% at o.3 mg/kg and 74% at 1 mg/kg dose (Table 2-2 and FIG. 2A).

TABLE 2-2
Percent A_{2A} receptor occupancy by PNQ-370 in rat
brain striatum after oral dosing at 3 hours

% Receptor Occupancy ± SEM

| | | |
|---|---|---|
| Dose | PNQ-370 | Preladenant |

| | | | |
|---|---|---|---|
| 0.1 | mg/kg | 75 ± 1.30 | 51 ± 0.88 |
| 0.3 | mg/kg | 80 ± 1.59 | 56 ± 4.75 |
| 1 | mg/kg | 91 ± 0.72 | 74 ± 1.47 |

With regard to the time course study, after oral dosing of rats with compounds at 1 mg per kg, PNQ-370 showed receptor occupancy of 85% at 3 hours, 76% at 6 hours, 58% at 12 hours, 44% at 18 hours and 11% at 24 hours as compared to vehicle control group (specific binding) which was considered as zero percent occupancy. Preladenant after oral dosing of rats at 1 mg per kg showed receptor occupancy of 65% at 3 hours, 33% at 6 hours, 39% at 12 hours, 17% at 18 hours and 0.9% at 24 hours (Table 2-3 and FIG. 2B). Values represent the mean±SEM (Tables 2-2 and 2-3, FIGS. 2A-2B).

TABLE 2-3
Time course of percent A_{2A} receptor occupancy by
PNQ-370 in rat brain striatum after oral dosing

% Receptor Occupancy ± SEM

| | | |
|---|---|---|
| Time after | PNQ-370, | Preladenant, |
| dosing | 1 mg/kg, PO | 1 mg/kg, PO |

| | | | |
|---|---|---|---|
| 3 | hrs | 86 ± 1.2 | 65 ± 1.3 |
| 6 | hrs | 76 ± 0.6 | 33 ± 2.2 |
| 12 | hrs | 58 ± 2.9 | 39 ± 2 |
| 18 | hrs | 44 ± 0.5 | 17 ± 3.6 |
| 24 | hrs | 11 ± 3.5 | 0.9 ± 0 |

### Example 3

### Potency: Binding to Human A_{2A} Adenosine Receptor

This study investigated the binding potency of PNQ-370 on human A_{2A} adenosine receptor (AdoR) in plasma membrane preparations from HEK-293 cells over-expressing human A_{2A} AdoR in an in vitro 96-well plate assay.

PNQ-370 was prepared as stock solution of 10 mM in dimethyl sulphoxide (DMSO). All further dilutions of PNQ-370, unless specified, were made in DMSO.

Cloning and expression of human A_{2A} AdoR in HEK-293 cells: Human A_{2A} AdoR was cloned and stably expressed in HEK-293 cells (clone #01) as follows. Human A_{2A} adenosine receptor was PCR amplified from expression ready clones in pCMV6-XL5 vector (OriGene Technologies, Catalog #SC119757). PCR amplification was carried out using the PCR high fidelity supermix (Finnzymes, Catalog #F5302) according to the manufacturer's protocol, using the following primers:

Forward Primer:
(SEQ ID NO: 1)
5′ CTT GCT AGC ACC ACC ATG CCC ATC ATG GGC TCC TCG 3′
Reverse Primer:
(SEQ ID NO: 2)
5′ CTG AAG CTT TCA GGA CAC TCC TGC TCC 3′

The PCR products were purified using a QIAquick PCR purification kit (Qiagen Inc.) and digested using the restriction enzymes NheI and HinDIII. The restriction digested products were ligated to pcDNA 3.1(+) (Invitrogen life technologies, Catalog #V790-20). The ligated products were transformed into DH5a bacterial strain and selected for Ampicillin resistance. The DNA sequence of the selected clones was confirmed by DNA sequencing.

Confirmed clones were transfected in HEK-293 cell line using lipofectamine. Selection pressure of 1 mg/ml G418 was applied 48 hours post transfection. The transfected HEK-293 cells were subsequently grown in medium containing 1 mg/mL G418 and the resistant colonies were selected for further expansion and analysis.

Culture of human A_{2A}-HEK-293 (clone #01) cells: For routine maintenance, the cells were trypsinized in 0.25% Trypsin-EDTA solution (Sigma) at 37° C., washed in incomplete DMEM (Invitrogen) and reseeded in complete DMEM supplemented with 10% FBS (Gibco), 1 mg/mL G418, 100 U/mL penicillin and 100 μg/mL streptomycin. Cells were routinely maintained at 37° C. in 5% CO2. Cells were regularly split and maintained at no more than 80% cell density at all times.

Freezing of human A_{2A}-HEK-293 (clone #01) cells: Healthy and log phase cells were harvested in 0.25% trypsin-EDTA solution and washed with incomplete DMEM at 1500 rpm for 5 min at room temperature. The cells were resuspended in freezing media (20% FBS, 10% DMSO in DMEM) at a density of 1×106 cells/mL and frozen slowly at the rate of 1° C./min. The frozen stocks were finally stored in liquid nitrogen.

Revival of human A_{2A}-HEK-293 (clone #01) cells: Frozen stocks of cells were thawed quickly at 37° C. and dispersed drop wise in 10 mL incomplete DMEM. The cells were washed twice in incomplete DMEM at 1500 rpm for 5 min at room temperature. The cells were seeded in DMEM (supplemented with 20% FBS, 1 mg/mL G418 and 100 U/mL penicillin and 100 μg/mL streptomycin) in 25 cm2 culture flasks and incubated at 37° C. in 5% CO₂. The cells were trypsinized and passaged further in DMEM (supplemented with 10% FBS, 1 mg/mL G418 and 100 U/mL penicillin and 100 μg/mL streptomycin) for routine maintenance.

Plasma membranes were prepared from the cells over-expressing human A_{2A} AdoR for use in the radioligand binding assays. Briefly, cells were harvested in 0.25% trypsin-EDTA and washed once in PBS followed by two washes in wash buffer (150 mM NaCl, 1 mM EDTA, 50 mM Tris, pH 7.4) at 1500 rpm for 5 minutes at room temperature. The cell pellet was incubated in sonication buffer (1 mM EDTA, 5 mM Tris, pH 7.4) for 10 min at 4° C. followed by sonication on ice for 6 minutes. Unlysed cells and debris, obtained as pellet on centrifugation (Eppendorf) at 1000×g for 10 minutes at 4° C. pellet, were discarded. The supernatant was centrifuged (Hitachi) at 49,000×g for 45 minutes at 4° C. The protein pellet was resuspended in sonication buffer supplemented with 1 U/mL adenosine deaminase (ADA, Roche) and incubated for 30 minutes at room temperature. The protein was washed twice with sonication buffer at 49,000×g for 45 minutes at 4° C. and the protein pellet was resuspended in 50 mM Tris, pH 7.4 supplemented with 1 U/mL ADA and 10% sucrose. The protein was estimated by Bradford assay and aliquots were stored at −80° C.

Radioligand binding assays were carried out to determine the binding potency of PNQ-370 for human A_{2A} AdoR. Competition assays were started by mixing 1 nM [3H]-ZM-241385 (human A_{2A} AdoR specific antagonist, American Radiochemicals) with various concentrations of test compounds and 10 μg membrane protein in reaction buffer (50 mM Tris pH 7.4, 1 mM EDTA, supplemented with 1 U/mL ADA). The assay reactions were incubated for 90 minutes at room temperature with constant shaking and stopped by filtration using 96-well-plate harvester (Molecular Devices). Unbound ligand was removed by washing four times with ice cold 50 mM Tris (pH 7.4). Non-specific binding was determined in the presence of 100 μM 5′-N-ethylcarboxamidoadenosine (NECA, Sigma). Radioligand binding was read using a MicroBeta TriLux liquid scintillation counter (Perkin Elmer), and the binding affinity of test compound (i.e. Ki value) was calculated using GraphPad Prism software.

Results are shown in Table 3-1 and FIGS. 3A-3C. Assay standard SCH-442416, known antagonist to human A_{2A} AdoR, binds at IC50 of 6.1±2.6 nM (Ki=3.1±1.4 nM). PNQ-370 binds to human A_{2A} AdoR at IC50 of 1.2±0.45 nM (Ki=0.60±0.24 nM). Preladenant binds to human A_{2A} AdoR at IC50=2.7±0.4 nM (Ki=1.3±0.2 nM) (n=6). SYN-115 binds to human A_{2A} AdoR at IC50=18.7±6.4 nM (Ki=9.9±3.5 nM) (n=4). Values represent mean±SEM from 6 independent experiments, each carried out in triplicates. In Table 3-1, data from each independent assay (Study No. 1-6) are represented as mean percent specific binding relative to vehicle control.

TABLE 3-1
Activity of PNQ-370 on human A_{2A} AdoR in
competitive radioligand binding assay

| Study No | IC₅₀ (nM) | Ki (nM) |
|---|---|---|
| 1 | 0.3 | 0.1 |
| 2 | 3.1 | 1.6 |
| 3 | 0.3 | 0.2 |
| 4 | 1.7 | 0.9 |
| 5 | 1.1 | 0.6 |
| 6 | 0.4 | 0.2 |
| Mean ± SEM | 1.2 ± 0.45 nM | 0.60 ± 0.24 nM |

### Example 4

### Evaluation of Binding Off-Rates on PNQ-370 on Human A_{2A} Adenosine Receptor

This study was performed to determine whether the longer brain occupancy of PNQ-370 observed in Example 2 was a function of increased residence time of PNQ-370 at the A_{2A} AdoR. In this study, binding assays were carried out with membranes over-expressing human A_{2A} AdoR to measure the Koff of PNQ-370 and preladenant, SYN-115 and MRS-1191 (an A₃ AdoR antagonist, >300-fold selective over A_{2A} AdoR).

PNQ-370, preladenant and SYN-115 were prepared as stock solutions of 10 mM in dimethyl sulphoxide (DMSO. All further dilutions, unless specified, were made in DMSO.

Commercially available plasma membrane fractions of HEK-293 cells over-expressing human A_{2A} AdoR were obtained from Perkin Elmer (Catalogue #RBHA2AM400UA) and used for the study.

The radioligand binding assays were carried out with 5 μg/well/100 μL membranes over-expressing human A_{2A} AdoR in reaction buffer (50 mM Tris, pH 7.4, 1 mM EDTA, supplemented with 1 U/mL adenosine deaminase) on 96-well multiscreen filter-plates (Millipore). The incubation steps were carried out at room temperature with constant shaking. Washing off of unbound ligand or antagonist was achieved by adding 100 μL cold reaction buffer (without adenosine deaminase) and centrifugation at 800×g (Eppendorf) for 1 minute at room temperature. Non-specific binding of radioligand was determined in presence of 100 μM 5′-N-ethylcarboxamidoadenosine (NECA, Sigma).

On-rate Determination: Preliminary binding assays were carried out for radioligand [3H]-ZM-241385 (2 nM, KD concentration, specific for A_{2A} AdoR) to determine the on-rate that allows the saturation of the total A_{2A} AdoR available. The approximate on-rates for the test compounds (at respective IC₈₀ concentrations, based on the IC₅₀ generated from in-house competitive radioligand binding assays) were also determined in a separate assay using 2 nM [3H]-ZM-241385.

Determination of Kon for ZM-241385: A_{2A} AdoR membranes (5 μg/well/100 μl in 96 well multiscreen filter-plates) were incubated with 2 nM [3H]-ZM-241385 in reaction buffer (50 mM Tris, pH 7.4, 1 mM EDTA, 1 U/ml ADA) for increasing time at room temperature with constant shaking. The reaction was stopped and unbound ligand removed by adding 100 μl ice cold reaction buffer (without ADA) and centrifugation at 800×g at room temperature. Ligand bound to the membrane on filter-plate was determined using Microbeta Trilux liquid scintillation counter. Non-specific binding was determined using 100 μM NECA. The data obtained was plotted using GraphPad Prism software. Based on the experiment, Kon for [3H]-ZM-241385 was estimated to be 15 minutes and used as the minimum incubation time for the radioligand in off-rate experiments.

Determination of Kon for PNQ-370, Preladenant and SYN-115: A_{2A} AdoR membranes (5 μg/well/100 μl in 96 well Multiscreen filter-plates) were incubated with antagonists PNQ-370 (6 nM), preladenant (12 nM) or SYN-115 (100 nM) for increasing time (0 min-60 min) in reaction buffer 96-well Multiscreen filter plates at room temperature with constant shaking. The unbound antagonists were removed by centrifugation at 800×g at room temperature and washing once with ice cold reaction buffer (without ADA). Membranes were then incubated with 2 nM [3H]-ZM-241385 for 15 minutes at room temperature with constant shaking. The reaction was finally stopped and unbound radioligand was removed by adding 100 μl ice cold reaction buffer (without ADA) followed by centrifugation at 800×g at room temperature. Bound ligand was determined using Microbeta Trilux liquid scintillation counter. Non-specific binding was determined using 100 μM NECA. The data obtained was plotted using GraphPad Prism software. Based on the experiment, the Kon for test compounds was estimated to be 10 minutes for PNQ-370 and preladenant and 15 min-20 min for SYN-115 and used as the minimum pre-incubation time with test compounds for off-rate experiments.

Off-rate Determination: The assay to determine the off-rates for test compounds was carried out as follows. The membranes were incubated with PNQ-370 (6 nM), preladenant (60 nM) and SYN-115 (200 nM) for 15 min or 20 min (based on the on-rates determined as described above). MRS-1191, an A₃ AdoR selective antagonist, was used at 300 nM (10 fold the IC50 on A₃ AdoR). The compounds were washed off and membranes were then incubated for varying time durations in reaction buffer (0 hour-10 hours) with a washing step included every one hour and a final wash at 30 minutes. Membranes were finally incubated with 2 nM [3H]-ZM-241385 for 15 minutes followed by washing thrice with reaction buffer (without ADA) to remove the unbound radioligand. After 1 hour of drying the membranes at 65° C., bound radioligand was determined using MicroBeta TriLux liquid scintillation counter (Perkin Elmer). [3H]-ZM-241385 retained on the membrane was indicative of A_{2A} AdoR sites not occupied by the test compounds. Data obtained was plotted using GraphPad Prism software. A schematic representation of the protocol is shown in FIG. 4A.

Results from two separate studies are shown in FIGS. 4B-4C. PNQ-370 has a long Koff on human A_{2A} AdoR and even after 10 hours of repeated washing, the compound does not appreciably dissociate from the receptor. In contrast, preladenant shows a steady rate of decline in binding, indicating that it has a quicker off-rate compared to PNQ-370. These observations are consistent with those seen in the brain receptor occupancy assay.

SYN-115 appears to have significantly different properties compared to PNQ-370 and preladenant, as observed with its much faster Koff (FIGS. 4B-4C). Almost 100% displacement of SYN-115 binding by ZM-241385 is observed within 1 hour. Despite using a saturating dose and longer pre-incubation with SYN-115, 100% inhibition of radioligand binding even at the earliest time point evaluated was not observed.

MRS-1191, a selective A₃ AdoR antagonist used as a negative control (based on its selectivity against A_{2A} AdoR), does not affect the binding of [3H]-ZM-241385 at any time point evaluated (FIGS. 4B-4C).

### Example 5

### Schild Regression Analysis of PNQ-370 on Human A_{2A} Adenosine Receptor

PNQ-370, preladenant and SYN-115 were prepared as stock solutions of 10 mM in dimethyl sulphoxide (DMSO). All further dilutions, unless specified, were made in DMSO.

Human A_{2A} AdoR was cloned and stably expressed in HEK-293 cells (clone #01) and regularly maintained, as described in Example 3. Log phase cultures, seeded overnight at 80% confluency, were used for the study.

HTRF-cAMP assays: A_{2A} AdoR is a GPCR that signals through Gs-mediated increase in the secondary messenger, cAMP. To determine the Schild slope and KB value for PNQ-370 in comparison to preladenant and SYN-115, the effect of different concentrations of antagonists on the concentration response curve of agonist (4-((2-carboxymethyl) phenyl) ethylamino)-5′-N-ethylcaboxamidoadenosine (CGS-21680) for activation of A_{2A} AdoR was evaluated using a homogenous time resolved fluorescence (HTRF)-cyclic adenosine monophosphate (cAMP) assay (Cis-bio, Cat #62AM5PEB) according to the manufacturer's recommended protocol, with minor modifications. Briefly, 500× concentrations of test compounds (PNQ-370, preladenant and SYN-115) and 100× concentrations of CGS-21680 (Tocris), specific agonist of A_{2A} AdoR, were prepared by serial dilution in DMSO and corresponding 4× dilutions were prepared in DMEM supplemented with 1 U/mL adenosine deaminase (ADA, Roche) and 20 μM rolipram (Sigma).

A 24 hour culture of HEK-293 cells over-expressing human Av AdoR was treated with 1 U/mL of ADA in Dulbecco's modified eagle medium (DMEM) supplemented with 10% FBS, for 90 minutes at 37° C. and 5% CO₂. Cells were harvested in dissociation buffer (Sigma) and washed in complete DMEM (supplemented with 10% FBS) followed by incomplete DMEM at 1300 rpm for 5 minutes. Cells were distributed at a density of 3750 cells/25 μL/well in DMEM (supplemented with 1 U/ml ADA) and incubated with test compounds (varying concentrations) for 15 minutes. Cells were then treated with different concentrations of CGS-21680 for 30 minutes. All treatments were carried out with continuous mixing at room temperature. 1% DMSO was used as vehicle. The contents of the wells were mixed thoroughly and 10 μL of each reaction mix was transferred to the 384-well plate.

The levels of cAMP were detected and estimated as per manufacturer's instruction using the Cisbio HTRF cAMP kit. The FRET between d2-labeled cAMP and cryptate labeled anti-cAMP antibody was read in HTRF mode in a Flex Station III (Molecular Devices) using an excitation maximum of 313 nm and emission maxima of 620 nm and 665 nm. Data was analyzed using GraphPad Prism software to determine EC50 values for the agonist in presence or absence of different test compounds at varying concentrations. The values were then used to calculate dose ratio (DR) and log (DR-1) was plotted against test compound concentrations to generate Schild plot and calculate Schild slope, pA2 and K_{B} values using GraphPad Prism. Results are shown in FIGS. 5A-5F and Table 5-1.

TABLE 5-1
Effect of PNQ-370 on concentration response curve
of agonist-induced activation of human A_{2A} AdoR

| | Kb (nM) | | |
|---|---|---|---|
| Compound | Mean | ±SEM | n |
| PNQ-370 | 0.5 | 0.04 | 5 |
| Preladenant | 2.5 | 0.99 | 3 |
| SYN-115 | 0.8 | 0.3 | 3 |

### Example 6

### Scatchard Analysis of PNQ-370 on Human A_{2A} Adenosine Receptor

A Scatchard analysis in a competitive radioligand binding assay was performed to evaluate whether the differences in residence time were simply a function of Koff, or whether differences in the mechanism of binding may also be contributing factors.

PNQ-370, preladenant and SYN-115 were prepared as stock solutions of 10 mM in dimethyl sulphoxide (DMSO). All further dilutions, unless specified, were made in DMSO.

Commercially available plasma membrane fractions of HEK-293 cells over-expressing human A_{2A} AdoR were obtained from Perkin Elmer (Catalogue #RBHA2AM400UA). Human A_{2A} AdoR (5 μg/well/100 μL) membranes were incubated with increasing concentrations of A_{2A} AdoR antagonist ZM-241385 (supplemented with [3H]-labeled ZM-241385 for detection) in reaction buffer (50 mM Tris, pH 7.4, 1 mM EDTA, 1 U/mL ADA) for 90 min at 37° C. The reaction was stopped by filtration through glass fiber filtermats using a 96-well harvester (Molecular Devices). Unbound ligand was removed by washing thrice with ice cold 50 mM Tris, pH 7.4, thus allowing only bound ligand to be retained on the filtermats. The filtermats were dried for 1 hour at 65° C., and bound ligand was determined using a Microbeta Trilux Liquid Scintillation Counter (Perkin Elmer).

Bound vs. total ligand concentrations were determined from the ratio of total CPM (measuring the counts from the entire quantity of ligand added without any filtration step) to the CPM obtained post-filtration. Data obtained was plotted using GraphPad Prism software to calculate KD and Bmax for ZM-241385.

The binding mechanism of PNQ-370 was determined by evaluating its effect on the Scatchard plot of A_{2A} AdoR antagonist ZM-241385. Preladenant and SYN-115 were used as comparator compounds. Membranes expressing human A_{2A} AdoR (5 μg/well/100 μL) were incubated with increasing concentrations of A_{2A} AdoR antagonist ZM-241385 (supplemented with [₃H]-labeled ZM-241385 for detection) in presence or absence of PNQ-370, preladenant or SYN-115 in reaction buffer (50 mM Tris pH 7.4, 1 mM EDTA, 1 U/mL ADA) for 90 min at 37° C. The concentrations of PNQ-370, preladenant or SYN-115 were chosen based on the IC₅₀ of each compound (PNQ-370: 1 nM and 3 nM; preladenant: 3 nM and 10 nM; SYN-115: 10 nM and 30 nM). The reaction was stopped by filtration through glass fiber filtermats using a 96-well harvester (Molecular Devices). Unbound ligand was removed by washing thrice with ice cold 50 mM Tris, pH 7.4. The filtermats were dried for 1 hour at 65° C., and bound ligand was determined using a MicroBeta TriLux Liquid Scintillation Counter (Perkin Elmer). The data obtained was used to calculate Bound/Free ligand CPMs and plotted against Bound ZM-241385 concentrations (nM) using GraphPad Prism software.

Specific binding in nM was determined from the ratio of total counts (no filtration or removal of any ligand) to the counts retained on the membrane (after correcting for nonspecific binding). Results are shown in FIGS. 6A-6E and Table 6-1.

The dissociation contestant (K_{D}) and density (concentration) of receptors in a sample of tissue (Bₘₐₓ) for ZM-241385 were found to be 3.9 and 0.75 nM, respectively (FIGS. 6A-6B). In a Scatchard analysis, the slope of the line reflects the K_{D}, and the intercept on the X-axis when Y=0 reflects the Bmax. As seen FIGS. 6C-6E competition with PNQ-370, preladenant and SYN-115 changes the K_{D} of ZM-241385 at the human A_{2A} AdoR receptor, without appreciably changing the Bmax. These results indicate that the binding of PNQ-370 as well as preladenant and SYN-115 is competitive with respect to ZM-241385.

TABLE 6-1
Bmax (nM) of AM-241385 (nM) in presence
of PNQ-370, preladenant and SYN-115.

| Compound | Bₘₐₓ (nM) |
|---|---|
| No compound | 0.69 |
| 1 nM PNQ-370 | 0.78 |
| 3 nM PNQ-370 | 1.05 |
| 3 nM Preladenant | 0.73 |
| 10 nM Preladenant | 0.81 |
| 10 nM SYN-115 | 0.78 |
| 30 nM SYN-115 | 0.87 |

### Example 7

### Functional Assay

This study investigated the functional activity of test compound PNQ-370 on (4-((2-carboxymethyl) phenyl) ethylamino)-5′-N-ethylcaboxamidoadenosine (CGS-21680)-induced activation of human A_{2A} AdoR in HEK-293 cells overexpressing A_{2A} AdoR in an in vitro multi-well plate assay.

PNQ-370 was prepared as stock solution of 10 mM in dimethyl sulphoxide (DMSO). All further dilutions of PNQ-370, unless specified, were made in DMSO.

Human A_{2A} AdoR was cloned and stably expressed in HEK-293 cells (clone #01) and regularly maintained, as described in Example 3. Log phase cultures, seeded overnight at 80% confluency, were used for the study.

HTRF-cAMP assays: A_{2A} AdoR is a G protein coupled receptor (GPCR) that signals through Gs mediated increase in secondary messenger, cAMP. The effect of PNQ-370 on agonist-induced activation of human A_{2A} AdoR was evaluated using a homogenous time resolved fluorescence (HTRF)-cyclic adenosine monophosphate (cAMP) assay (Cis-bio, Cat #62AM5PEB) according to the manufacturer's recommended protocol, with minor modifications. Briefly, 100× concentrations of test compound were prepared by serial dilution in DMSO and corresponding 4× dilutions were prepared in DMEM supplemented with 1 U/mL adenosine deaminase (ADA, Roche) and 20 μM rolipram (Sigma).

A 24 hour culture of HEK-293 cells over-expressing human A_{2A} AdoR was treated with 1 U/mL of ADA in Dulbecco's modified eagle medium (DMEM) supplemented with 10% fetal bovine serum (FBS) for 90 minutes at 3 7° C. and 5% CO₂. Cells were harvested in dissociation buffer (Sigma) and washed in complete DMEM (supplemented with 10% FBS) followed by incomplete DMEM at 1300 rpm for 5 minutes. Cells were distributed at a density of 3750 cells/25 μL/well in DMEM (supplemented with 1 U/ml ADA) and incubated with increasing concentrations of test compound for 15 minutes, followed by treatment with specific A_{2A} AdoR agonist CGS-21680 (final concentration 10 nM, Tocris) for 30 minutes (1% DMSO was used as vehicle). All treatments were carried with continuous mixing at room temperature. The contents of the wells were mixed thoroughly and 10 μL of each reaction mix was transferred to the 384-well plate.

The levels of cAMP were detected and estimated as per manufacturer's instruction using the Cisbio HTRF cAMP kit. The FRET between d2-labeled cAMP and cryptate labeled anti-cAMP antibody was read in HTRF mode in a Flex Station III (Molecular Devices) using an excitation maximum of 313 nm and emission maxima of 620 nm and 665 nm. Data was analyzed using GraphPad Prism software to determine IC₅₀ and Ki values.

Cells over-expressing human A_{2A} AdoR were treated with increasing concentrations of PNQ-370 in presence of 10 nM A_{2A} AdoR CGS-21680 (KD=2 nM). Change in cAMP levels was measured using an HTRF-based competitive immunoassay. The data from individual experiments is presented in Table 7-1.

PNQ-370 inhibits CGS-21680-induced activation of human A_{2A} AdoR at IC₅₀ of 0.4±0.1 nM (Ki=0.062±0.02 nM). Assay standard preladenant, known antagonist of human A_{2A} AdoR, inhibits CGS-21680-induced A_{2A} AdoR activity at IC₅₀ of 5.1±2.4 nM (Ki=0.7±0.3 nM). Values represent mean±SEM from 5 independent experiments, each carried out in triplicates (Table 7-1, FIG. 7A).

TABLE 7-1
Effect of PNQ-370 on agonist-induced
activation of human A_{2A} AdoR

| Study No | IC₅₀ (nM) | Kᵢ (nM) |
|---|---|---|
| 1 | 0.3 | 0.04 |
| 2 | 0.7 | 0.1 |
| 3 | 0.3 | 0.05 |
| 4 | 0.7 | 0.1 |
| 5 | 0.13 | 0.02 |
| Mean ± SEM | 0.4 ± 0.1 nM | 0.06 ± 0.02 nM |

Preladenant and SYN-115 were evaluated as comparator compounds in separate experiments. Preladenant inhibits CGS-21680-induced A_{2A} AdoR activation at IC₅₀=5.2±1.2 nM (Ki=0.7±0.2 nM) (n=4) (FIG. 7B). SYN-115 inhibits CGS-21680-induced A_{2A} AdoR activation at an IC₅₀=7.0±3.1 nM (Ki=0.9±0.4 nM) (n=5) (FIG. 7C).

While a number of exemplary aspects and embodiments have been discussed above, those of skill in the art will recognize certain modifications, permutations, additions and sub-combinations thereof. It is therefore intended that the following appended claims and claims hereafter introduced are interpreted to include all such modifications, permutations, additions and sub-combinations as are within their true spirit and scope.

1. A method for treating a solid tumor, comprising:
orally administering Compound 1 to a subject in need at a dose of between about 1-30 mg per day, wherein Compound 1 is 5-amino-3-[2-[4-[2-fluoro-4-(2-methoxyethoxy)phenyl]piperazin-1-yl]ethyl]-8-(2-furyl)-1-methyl-[1,2,4]triazolo[5, 1-f]purin-2-one.

2. The method of claim 1, further comprising determining an A_{2A} receptor expression level of the solid tumor prior to said orally administering.

3. The method of claim 1, wherein orally administering comprises orally administering as a monotherapy.

4. The method of claim 3, wherein the method further comprises, before, after or concurrent with said administering surgical resection of the solid tumor.

5. The method of claim 3, wherein the method further comprises, before, after or concurrent with said administering radiation treatment.

6. The method of claim 1, wherein said orally administering comprises orally administering the dose as a total daily dose divided into two portions.

7. The method of claim 1, wherein the solid tumor is associated with breast cancer or colon cancer.

8. A method for treating a solid tumor, comprising:
orally administering to a subject in need 5-amino-3-[2-[4-[2-fluoro-4-(2-methoxyethoxy)phenyl]piperazin-1-yl]ethyl]-8-(2-furyl)-1-methyl-[1,2,4]triazolo[5, 1-f]purin-2-one (Compound 1) at a dose of between about 0.5-15 mg given twice daily, wherein said orally administering provides a concentration of Compound 1 in a solid tumor 10 fold higher than a concentration in the plasma.

9. The method of claim 8, wherein the concentration of Compound 1 in a solid tumor is 10 fold higher than a concentration in the plasma within about 2 hours after oral administration.

10. The method of claim 8, wherein the concentration of Compound 1 in a solid tumor is 10 fold higher than a concentration in the plasma for between about 1-4 hours after oral administration.

11. The method of claim 8, wherein the orally administering is with food.

12. The method of claim 8, wherein the solid tumor is castrate-resistant prostate cancer, non-small cell lung cancer, head and neck cancer, or renal cell cancer.

13. The method of claim 12, wherein the non-small cell lung cancer is squamous cell cancer.

14. A method for treatment, comprising:
orally administering 5-amino-3-[2-[4-[2-fluoro-4-(2-methoxyethoxy)phenyl]piperazin-1-yl]ethyl]-8-(2-furyl)-1-methyl-[1,2,4]triazolo[5, 1-f]purin-2-one (Compound 1), and
administering a second compound that has activity to inhibit/antagonize (i) an adenosine A2B receptor or (ii) a checkpoint programmed death 1 (PD-1) receptor.

15. The method of claim 14, wherein said administering the second compound comprises administering concurrently with or subsequently to administering Compound 1.

16. The method of claim 14, wherein said administering the second compound comprises administering enterally or parenterally.

17. The method of claim 14, wherein the second compound is an antagonist of the A2B receptor.

18. The method of claim 17, wherein the second compound is 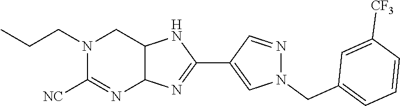 [SMILES: CCCN1CC2NC(c3cnn(Cc4cccc(C(F)(F)F)c4)c3)=NC2N=C1C#N]
6-Oxo-1-propyl-8-[1-(3-trifluoromethyl-benzyl)-1H-pyrazol-4-yl]-6, 7-dihydro-1H-purine-2-carbonitrile.

19. The method of claim 14, wherein the second compound is an inhibitor of the PD-1 receptor.

20. The method of claim 19, wherein the second compound is an anti-PD-1 receptor antibody.

21-27. (canceled)


SEQUENCE LISTING (2 sequences)

SEQ ID NO: 1 - DNA, 36 bp, synthetic construct
  source 1..36: mol_type = other DNA
cttgctagcaccaccatgcccatcatgggctcctcg

SEQ ID NO: 2 - DNA, 27 bp, synthetic construct
  source 1..27: mol_type = other DNA
ctgaagctttcaggacactcctgctcc